(19) United States
(12) Patent Application Publication
(10) Pub. No.: US 2026/0033979 A1
(43) Pub. Date: Feb. 5, 2026

(54) Ostomy Garment System with Integrated Support and Containment Features

(71) Applicant: Hallie Ammacher, Carlsbad, CA (US)

(72) Inventor: Hallie Ammacher, Carlsbad, CA (US)

(21) Appl. No.: 18/794,125

(22) Filed: Aug. 5, 2024

(51) Int. Cl. A61F 5/449; A41D 31/04

(52) U.S. Cl. CPC A61F 5/449; A41D 31/04

(57) ABSTRACT

An ostomy garment system comprising a pair of shorts with a waist portion having a thick strip for securing a top dome over a stoma, and a leg portion with a first attachment point. The top dome, made of plastic with ventilation holes and memory foam pads, protects the stoma. A bottom portion, coupled to the leg portion via a second attachment point, holds an ostomy bag and includes a plastic cup-shaped structure, an absorbent material layer, and a drain cap. The system provides improved support, leakage reduction, comfort, and convenience for ostomy bag users. The invention also discloses a method of using the ostomy garment system.

### BACKGROUND

### Field of the Invention

The present invention relates generally to the field of ostomy support garments and, more specifically, to a garment designed to provide protection, support, and comfort for individuals with ostomies.

### Description of the Related Art

Individuals who have undergone ostomy surgery, which involves creating an opening in the abdominal wall to allow waste to exit the body, face unique challenges in their daily lives. These challenges include discomfort, lack of protection for the stoma (the surgically created opening), and difficulty managing ostomy bags, which are attached to the stoma to collect waste.

Various garments have been developed to address these issues, such as the ostomy garment disclosed in U.S. Pat. No. 5,135,520 to Beaupied. This garment features a pair of crisscross pocket forming panels configured to lie behind an ostomy device, with the panels being releasably joined together at their top portions over a range of relative positions to provide adjustable support and tension.

While garments like the one described in Beaupied offer some benefits, there remains a need for a more comprehensive solution that integrates stoma protection, ostomy bag support, and comfortable wear into a single, easy-to-use garment. The present invention, referred to as “Tom Shorts,” aims to fulfill this need by providing a novel ostomy support garment that prioritizes the comfort, security, and discretion of ostomy patients.

### SUMMARY

The present invention is directed to an ostomy garment system that provides protection, support, and comfort for individuals with ostomies. The system includes a pair of shorts with a waist portion having a thick strip at the stoma level for securing a top dome, and a leg portion with a first attachment point. The top dome is configured to cover and protect the stoma, and includes an arch-shaped structure made from a plastic material, a plurality of air holes for ventilation, and one or more memory foam pads for comfort. The bottom portion is configured to hold an ostomy bag and includes a cup-shaped structure made from a plastic material, a material layer for reducing leakage noticeability, a second attachment point for coupling with the first attachment point on the leg portion to secure the ostomy bag against the user's leg, and a drain cap for emptying the ostomy bag.

In some embodiments, the space between the top dome and the bottom portion includes an opening to allow passage of the ostomy bag. The first and second attachment points may comprise a hook and loop fastener. The waist portion of the shorts may include an adjustable waistband for a secure and comfortable fit, and the shorts may be made from a flexible and breathable material. The material layer on the bottom portion may be an absorbent fabric for wicking moisture away from the skin. The drain cap may be removable and resealable for easy emptying and cleaning. The system may further include a deodorizing filter on the top dome for reducing odors, and the memory foam pads may be removable and replaceable for customizing comfort and support. The shorts may also include a pocket for storing ostomy care accessories.

The invention also provides a method of using the ostomy garment system. The method includes providing the ostomy garment, positioning the shorts on the user with the thick strip at the stoma level, attaching the ostomy bag to the top dome such that it passes through the opening to the bottom portion, securing the ostomy bag to the leg portion using the attachment points, emptying the bag as needed using the drain cap, and removing the garment by detaching the bag and removing the shorts. In some embodiments, the top dome may include an antimicrobial coating to reduce infection risk, and the shorts may be made of a stretchable, breathable fabric for comfort and flexibility.

Additional features and advantages of the invention will be set forth in the description which follows, and in part will be obvious from the description, or may be learned by the practice of the invention. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 is a front view of an embodiment of an ostomy garment system according to the present invention.

FIG. 2 is an exploded view of a top dome and bottom portion of an embodiment of the ostomy garment system of FIG. 1.

FIG. 3 is a flow diagram illustrating a method of using an embodiment of the ostomy garment system.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can”, “could”, “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

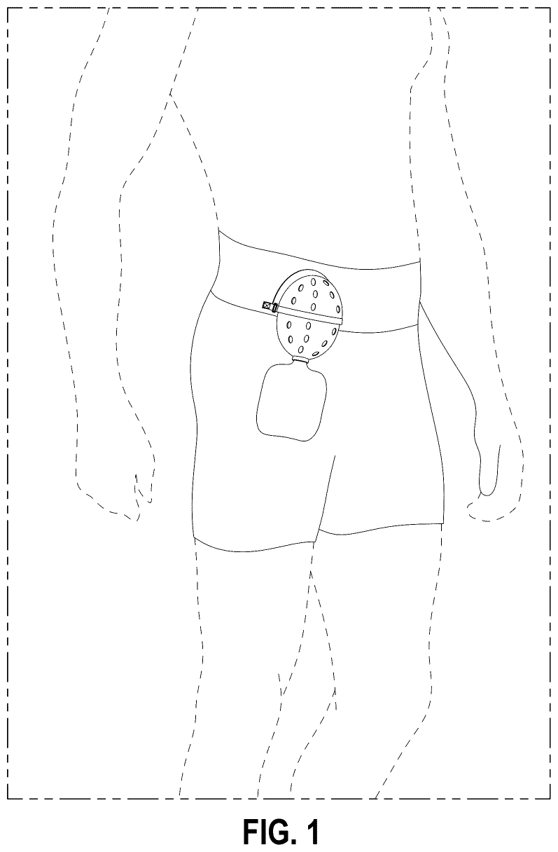

Referring to FIG. 1, an embodiment of an ostomy garment system 100 is shown. The ostomy garment system 100 includes a pair of shorts 110 configured to be worn by a user. In this embodiment, the shorts 110 comprise a waist portion 112 and a leg portion 114.

The waist portion 112 of the shorts 110 in this embodiment includes a thick strip 116 positioned at a stoma level of the user when the shorts 110 are worn. In some embodiments, the thick strip 116 may be adjustable to accommodate different body sizes and stoma positions. The thick strip 116 is configured for securing a top dome 130 at the stoma level.

In the present embodiment, the leg portion 114 of the shorts 110 includes a first attachment point 118. The first attachment point 118 may comprise one half of a hook and loop fastener. In this embodiment, the shorts 110 are made from a flexible, stretchable and breathable material to allow for comfort, movement and breathability for the user.

The top dome 130 in this embodiment is configured to cover and protect a stoma of the user. The top dome 130 has an arch-shaped structure 132 made from a plastic material. In the illustrated embodiment, the arch-shaped structure 132 includes a plurality of air holes 134 disposed thereon to provide ventilation to the stoma area. One or more memory foam pads 136 are disposed on an interior surface of the arch-shaped structure 132 to provide comfort against the user's skin around the stoma.

In the present embodiment, a bottom portion 140 is configured to hold an ostomy bag 150. The bottom portion 140 has a cup-shaped structure 142 made from a plastic material. A material layer 144 is disposed over the exterior of the cup-shaped structure 142 to help reduce the noticeability of any leakage from the ostomy bag 150.

The bottom portion 140 in this embodiment includes a second attachment point 146 configured to couple with the first attachment point 118 on the leg portion 114 of the shorts 110. The second attachment point 146 may comprise the mating half of the hook and loop fastener of the first attachment point 118. In this embodiment, coupling the first and second attachment points 118, 146 allows the ostomy bag 150 to be secured against the leg of the user and move with the motion of the user's leg.

In the illustrated embodiment, a drain cap 148 is disposed at the bottom of the cup-shaped structure 142. The drain cap 148 can be opened to allow the ostomy bag 150 to be emptied as needed.

In some embodiments, the shorts 110 may further include a pocket 120 disposed on an exterior surface thereof. The pocket 120 is configured for conveniently storing ostomy care accessories.

In use, the user positions the pair of shorts 110 such that the thick strip 116 at the waist portion 112 is located at the waist or stoma level. In this embodiment, the ostomy bag 150 is attached to the top dome 130 and passed through the opening between the top dome 130 and bottom portion 140. The bottom portion 140 is secured to the leg portion 114 by coupling the first and second attachment points 118, 146 so that the ostomy bag 150 is held securely against the leg and can move with the leg's motion. As needed, the drain cap 148 can be opened to empty the contents of the ostomy bag 150. To remove the ostomy garment system 100 in this embodiment, the ostomy bag 150 is detached from the top dome 130 and bottom portion 140, and the shorts 110 are removed from the user's body.

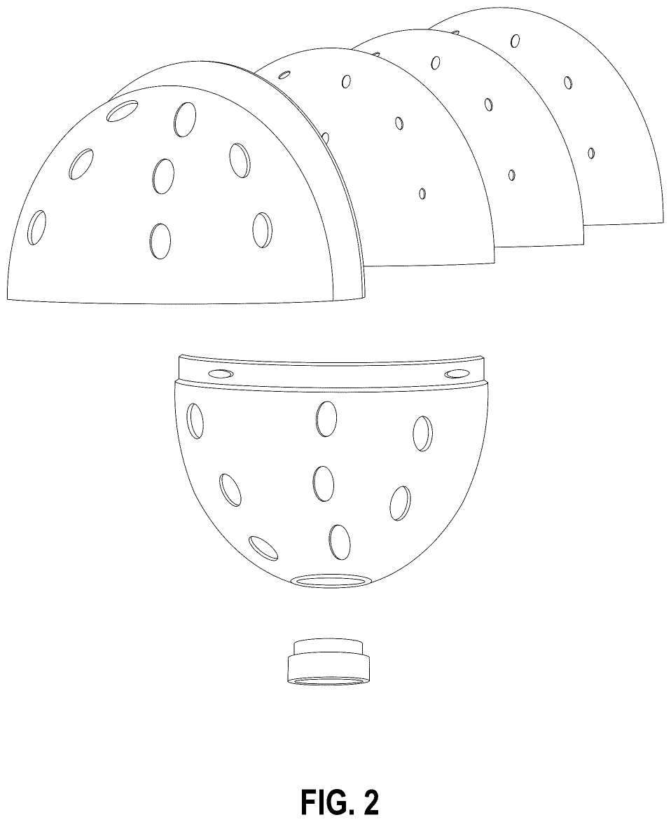

Referring now to FIG. 2, the top dome 130 and bottom portion 140 of an embodiment of the ostomy garment system 100 are shown in more detail.

In this embodiment, the top dome 130 includes the arch-shaped structure 132 made from a plastic material. The plurality of air holes 134 are disposed on the arch-shaped structure 132 to allow for ventilation and air circulation around the stoma. The one or more memory foam pads 136 are disposed on an interior surface 133 of the arch-shaped structure 132 to provide comfort and support to the stoma area. In some embodiments, the memory foam pads 136 may be removable and replaceable to allow for customization of the level of comfort and support.

In the present embodiment, an antimicrobial coating 138 is disposed on the interior surface 133 of the arch-shaped structure 132. The antimicrobial coating 138 helps reduce the risk of infection at the stoma site. A deodorizing filter 139 is also disposed on the top dome 130 in this embodiment to help reduce any odors associated with the stoma.

The bottom portion 140 of this embodiment includes the cup-shaped structure 142 made from a plastic material. The cup-shaped structure 142 is configured to hold an ostomy bag 150 (not shown in this figure). A material layer 144 is disposed over an exterior surface of the cup-shaped structure 142. In the present embodiment, the material layer 144 comprises an absorbent fabric that helps wick moisture away from the user's skin and reduces the noticeability of any leakage from the ostomy bag 150. In alternative embodiments, the material layer 144 may comprise a moisture-wicking material to further enhance comfort and reduce skin irritation.

In this embodiment, the second attachment point 146 is disposed on the bottom portion 140. As previously described, the second attachment point 146 is configured to couple with the first attachment point 118 on the leg portion 114 of the shorts 110 to secure the ostomy bag 150 against the user's leg. In the illustrated embodiment, the second attachment point 146 and first attachment point 118 may comprise mating hook and loop fasteners to provide secure but adjustable attachment.

A drain cap 148 is disposed at a bottom end of the cup-shaped structure 142 in this embodiment. The drain cap 148 is removable and resealable to allow for convenient emptying and cleaning of the ostomy bag 150. In some embodiments, the drain cap 148 may be configured to be operated with one hand for ease of use.

In the present embodiment, an opening 135 is provided between the top dome 130 and bottom portion 140. The opening 135 is configured to allow passage of the ostomy bag 150 from the stoma site to the cup-shaped structure 142 of the bottom portion 140. In alternative embodiments, a waterproof layer (not shown) may be disposed between the top dome 130 and the shorts 110 to prevent any leakage from the opening 135 onto the shorts 110.

In some embodiments, the top dome 130 and bottom portion 140 may be removable and replaceable from the shorts 110 and each other. This allows for easier cleaning and maintenance of the ostomy garment system 100.

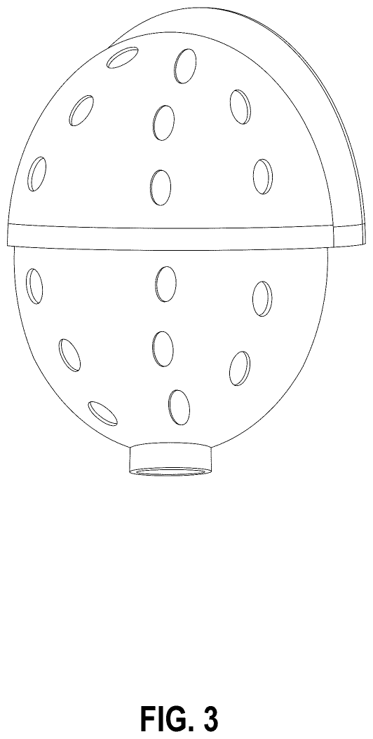

Referring to FIG. 3, a method 300 of using an embodiment of the ostomy garment system 100 is illustrated. The method 300 depicts the overall flow and sequence of using the ostomy garment system 100.

At step 301, the method 300 includes providing an embodiment of the ostomy garment system 100. The ostomy garment system 100 comprises the pair of shorts 110 having the waist portion 112 with the thick strip 116 at the stoma level and the leg portion 114 with the first attachment point 118. The ostomy garment system 100 further includes the top dome 130 attached to the thick strip 116 at the stoma level. In this embodiment, the top dome 130 has the arch-shaped structure 132 made of plastic with the plurality of air holes 134 and the one or more memory foam pads 136. An antimicrobial coating 138 may be disposed on an inner surface 133 of the top dome 130 to reduce the risk of infection. The ostomy garment system 100 also includes the bottom portion 140 attached to the first attachment point 118 on the leg portion 114. The bottom portion 140 has the cup-shaped structure 142 made of plastic, the material layer 144, the drain cap 148, and the second attachment point 146. The material layer 144 may comprise a moisture-wicking material to enhance comfort and reduce skin irritation.

At step 302, the method 300 includes positioning the pair of shorts 110 on the user such that the thick strip 116 at the waist portion 112 is located at the waist or stoma level. In some embodiments, the thick strip 116 may be adjustable to accommodate different body sizes and stoma positions. The pair of shorts 110 may be made of a stretchable, breathable fabric to provide comfort and flexibility during wear.

Step 303 involves attaching the ostomy bag 150 to the top dome 130 such that the ostomy bag 150 passes through the opening 135 between the top dome 130 and the bottom portion 140. A waterproof layer (not shown) may be provided between the top dome 130 and the pair of shorts 110 to prevent leakage.

At step 304, the ostomy bag 150 is secured to the first attachment point 118 on the leg portion 114 using the second attachment point 146 on the bottom portion 140. This allows the ostomy bag 150 to move with the motion of the user's leg and be held securely against the leg. The first attachment point 118 and second attachment point 146 may comprise hook and loop fasteners for secure and adjustable attachment.

Step 305 involves emptying the ostomy bag 150 as needed using the drain cap 148 on the bottom portion 140. The drain cap 148 may be configured to be operated with one hand for ease of use.

At step 306, the method 300 includes removing the ostomy garment system 100 by detaching the ostomy bag 150 from the first attachment point 118 on the leg portion 114 and the top dome 130, and removing the pair of shorts 110. In some embodiments, the top dome 130 and bottom portion 140 may be removable and replaceable to allow for cleaning and maintenance of the ostomy garment system 100.

Throughout the usage process, the plurality of air holes 134 in the top dome 130 allow for air circulation around the stoma, while the memory foam pads 136 and moisture-wicking material layer 144 enhance comfort for the user.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. An ostomy garment system comprising:
a pair of shorts comprising:
a waist portion having a thick strip at a stoma level for securing a top dome; and
a leg portion having a first attachment point;
a top dome configured to cover and protect a stoma, the top dome comprising:
an arch-shaped structure made from a plastic material;
a plurality of air holes disposed on the arch-shaped structure for ventilation; and
one or more memory foam pads disposed on an interior surface of the arch-shaped structure for providing comfort;
a bottom portion configured to hold an ostomy bag, the bottom portion comprising:
a cup-shaped structure made from a plastic material;
a material layer disposed over the cup-shaped structure for reducing leakage noticeability;
a second attachment point configured to couple with the first attachment point on the leg portion of the shorts for securing the ostomy bag against a leg of a user; and
a drain cap disposed on the cup-shaped structure for emptying the ostomy bag.

2. The ostomy garment system of claim 1, wherein the space between the top dome further comprises an opening disposed between the arch-shaped structure and the bottom portion, the opening configured to allow passage of the ostomy bag.

3. The ostomy garment system of claim 1, wherein the first attachment point and the second attachment point comprise a hook and loop fastener.

4. The ostomy garment system of claim 1, wherein the waist portion of the shorts further comprises an adjustable waistband for providing a secure and comfortable fit.

5. The ostomy garment system of claim 1, wherein the shorts are made from a flexible and breathable material to allow for movement and comfort.

6. The ostomy garment system of claim 1, wherein the material layer disposed over the cup-shaped structure of the bottom portion comprises an absorbent fabric for wicking moisture away from the skin.

7. The ostomy garment system of claim 1, wherein the drain cap is removable and resealable for easy emptying and cleaning of the ostomy bag.

8. The ostomy garment system of claim 1, further comprising a deodorizing filter disposed on the top dome for reducing odors associated with the stoma.

9. The ostomy garment system of claim 1, wherein the one or more memory foam pads are removable and replaceable for customizing the level of comfort and support provided to the stoma.

10. The ostomy garment system of claim 1, further comprising a pocket disposed on an exterior surface of the shorts for storing ostomy care accessories.

11. A method of using an ostomy garment, the method comprising:
providing an ostomy garment comprising:
a pair of shorts having a waist portion with a thick strip at a stoma level and a leg portion with a first attachment point;
a top dome attached to the thick strip at the stoma level, the top dome having an arch-shaped plastic structure with a plurality of air holes and one or more memory foam pads; and
a bottom portion attached to the first attachment point on the leg portion, the bottom portion having a cup-shaped plastic structure, a material layer, a drain cap, and a second attachment point;
positioning the pair of shorts on a user such that the thick strip at the stoma level is located at a waist or stoma level of the user;
attaching an ostomy bag to the top dome such that the ostomy bag passes through an opening between the top dome and the bottom portion;
securing the ostomy bag to the first attachment point on the leg portion using the second attachment point on the bottom portion, such that the ostomy bag moves with a motion of a leg of the user and is held securely against the leg;
emptying the ostomy bag as needed using the drain cap on the bottom portion; and
removing the ostomy garment by detaching the ostomy bag from the first attachment point on the leg portion and the top dome, and removing the pair of shorts.

12. The method of claim 11, wherein the top dome further comprises an antimicrobial coating on an inner surface configured to reduce the risk of infection.

13. The method of claim 11, wherein the pair of shorts is made of a stretchable, breathable fabric configured to provide comfort and flexibility during wear.

14. The method of claim 11, further comprising providing a waterproof layer between the top dome and the pair of shorts configured to prevent leakage.

15. The method of claim 11, wherein the drain cap on the bottom portion is configured to be operated with one hand for ease of use.

16. The method of claim 11, wherein the top dome and bottom portion are removable and replaceable to allow for cleaning and maintenance of the ostomy garment.

17. The method of claim 11, wherein the plurality of air holes in the top dome are configured to allow air circulation around the stoma.

18. The method of claim 11, wherein the material layer of the bottom portion comprises a moisture-wicking material configured to enhance comfort and reduce skin irritation.

19. The method of claim 11, wherein the thick strip at the stoma level is adjustable to accommodate different body sizes and stoma positions.

20. The method of claim 11, wherein the first attachment point on the leg portion and the second attachment point on the bottom portion comprise hook and loop fasteners configured for secure and adjustable attachment of the ostomy bag.
